(19) United States
(12) Patent Application Publication
(10) Pub. No.: US 2004/0077093 A1
(43) Pub. Date: Apr. 22, 2004

(54) Method and apparatus for the detection of the presence of a bacteria in the gastrointestinal tract of a subject

(75) Inventor: Lee Pan, Tampa, FL (US)

Correspondence Address: Mr. Joseph P. Reagen, Corporate Counsel, Renal Division, Baxter Healthcare Corporation, One Baxter Parkway, Deerfield, IL, 60015-4633, US

(73) Assignees: Baxter International Inc.; Baxter Healthcare S.A.

(21) Appl. No.: 10/617,008

(22) Filed: Jul. 10, 2003

Related U.S. Application Data
(60) Provisional application No. 60/395,268, filed on Jul. 12, 2002.

(51) Int. Cl. C12Q 1/04

(52) U.S. Cl. 436/37; 435/34

(57) ABSTRACT

The present invention generally relates to a method and an apparatus to detect the presence of a bacteria in a subject. More particularly, the method is directed toward the detection of a bacteria which is associated with catalyzing urea to carbon dioxide and ammonia when present in the gastrointestinal tract of a subject. The method comprises administering urea to a subject, obtaining a fluid sample from the subject after the administration of the urea and then determining the presence or amount of ammonia gas in the fluid sample. The apparatus facilitates ease of implementing the method and facilitates accuracy and speed of obtaining the test result.

### FIELD OF THE INVENTION

The present invention generally provides a method and an apparatus for the detection of a bacteria in a subject. More particularly, the method is directed to the detection of a bacteria associated with the catalytic breakdown of urea to carbon dioxide and ammonia in the gastrointestinal tract of a subject.

### BACKGROUND OF THE INVENTION

Gastrointestinal associated disorders are an important public health concern. In the United States alone, approximately twenty-five million Americans suffer from peptic ulcer disease with an annual incremental 500,000 to 800,000 new cases. Additionally, costs associated with treating these disorders are considerable.

Until recently, excessive gastric acidity and mental stress were thought to be the major pathophysiological reasons for the occurrence of gastrointestinal disorders. In 1982, however, a new spiral Gram-negative bacterium, later to be named Helicobacter pylori, was isolated from the gastric mucosa of significant numbers of patients inflicted with gastritis (Marshall et al, Med. J. Aust., 142(8): 439-44, 1985). Since these initial investigations, H. pylori and a recently discovered Helicobacter strain, Helicobacter heilmanii, have been shown to play an important role in causing such gastrointestinal disorders as peptic and gastric ulcers, gastric carcinoma, gastric lymphoma, gastritis, duodenitis, and esophagitis. In particular, these bacterium are estimated to have caused more than 90% of duodenal ulcers and up to 80% of gastric ulcers. Furthermore, H. pylori infected persons have a 2- to 6-fold increased risk of developing gastric cancer and mucosal-associated-lymphoid-type lymphoma.

Early detection of the presence of Helicobacter infection in the gastrointestinal tract improves an individual's prognosis. If detected early, H. pylori can be successfully treated with common antibiotics, such as penicillin or erythromycin, with no significant relapse in occurrence of disease. Moreover, early detection and immediate treatment by antibiotics is cost effective relative to other treatment regimes. For example, the cost of early treatment with antibiotics is a small fraction of the cost of surgery and post-surgical care. Thus, there is a great need for a reliable and simple method to diagnose the presence of H. pylori early in its infection cycle.

One method which has been employed for detecting the presence of H. pylori and disease conditions associated with it, requires the insertion of an endoscope into the stomach of a patient and withdrawal of a biopsy specimen for direct visual examination of the gastric mucosa tract. This method, however, is highly invasive, often causing significant patient discomfort, and requires trained personnel to carry out the procedure.

Another method for the detection of H. pylori infection requires collecting gas in the gastric cavity, and detecting in this gas ammonia and organic amines that are generated due to activities of the bacilli (see, e.g., U.S. Pat. No. 6,312,918). In this method, gas from the gastric cavity is led into the oral cavity by generating a vomiting-reflex, and the gas is collected by means of a metering suction pump which causes the gas to flow through a detection tube which changes color when ammonia and organic amines are present. Again, however, this technique is invasive, causes discomfort to the patient and is relatively expensive to perform.

Serological tests for H. pylori infection are somewhat less invasive. In these methods, a sample of blood is withdrawn and tested for the presence of IgA or IgG antibodies to H. pylori (see, e.g., U.S. Pat. No. 5,989,840). About twenty days from the time of infection, however, are required for antibodies against the bacterium to manifest themselves which can significantly compromise early detection. Also, antibodies can remain for 6-24 months after the bacteria have been eradicated, leading to a falsely positive result in about 10 to 15% of patients.

Breath tests have also been proposed for the detection of H. pylori infection. These tests exploit the fact that H. pylori produce and release urease, which catalyzes the degradation of urea into ammonia and carbon dioxide. For example, in one approach to a breath test (see, e.g., U.S. Pat. Nos. 4,830,010 and 6,067,989 and WO 97/26827), isotopically-labeled urea (e.g., 13C, 14C or 15N), in solid or liquid form, is orally ingested by the patient. If present, the bacteria convert the ingested urea to carbon dioxide and ammonia in the stomach. The concentration of isotopically labeled carbon, in the form of carbon dioxide, or nitrogen, in the form of ammonia, in a breath sample is then measured by mass spectrometry or a near infrared laser. Radioactive isotopes, however, have a relatively short half-life and raise safety and health issues for technicians as well as the patients. Non-radioactive isotopes possess other disadvantages; for example, the relative natural abundance of 13C is approximately 1% and thus, it is difficult to measure the amount of the isotope in a sample and the cost and complexity associated with the mass spectrometry pose significant drawbacks in any event.

In an alternative breath analysis method, a color changing indicator is used instead of a mass spectrometer or an infrared laser (see, e.g., WO97/30351). In this method, urea is administered to the subject and the subject's breath is thereafter analyzed for the presence of ammonia through the use of a composition which changes color when ammonia is present in the breath. Although it is disclosed that pH sensitive dyes may be used for this purpose, the preferred color indicator is a complex of a transition metal ion with the ammonia in an acidic environment. Complexes of the transition metal ions can be highly colored and can, therefore, form the basis of an indicator of the presence of ammonia. Disadvantageously, however, the metal ion, in addition to forming a complex with ammonia, also forms complexes with other substances present in the aqueous solution. These complexes also trigger a color change and therefore, can significantly bias the results of the test.

### SUMMARY OF THE INVENTION

Among the several aspects of the invention therefore, is provided a method and apparatus for the detection of H. pylori and other bacteria associated with the catalytic degradation of urea to ammonia and carbon dioxide in the gastrointestinal tract of a subject. Advantageously, the method is non-invasive, relatively inexpensive to perform, does not require the use of relatively expensive equipment, and does not provide false positives.

Briefly, therefore, one aspect of the present invention is a method for detecting in the gastrointestinal tract of a subject, the presence of a bacteria which when present in the gastrointestinal tract of the subject is associated with the catalytic degradation of urea to ammonia and carbon dioxide. The method comprises delivering a source of urea to the gastrointestinal tract of the subject, obtaining a fluid sample from the subject after the delivery of the urea source, contacting the fluid sample with a sensor, and optically detecting a color change in the sensor (which is indicative of the presence of ammonia in the fluid sample). The sensor comprises a polymeric material and a dye associated with the polymeric material, the dye having the capacity to become deprotonated and undergo a color change in the presence of ammonia. The contact conditions are controlled so that the sensor responds to the presence of ammonia in the fluid sample but not to the pH of the fluid sample by undergoing an optically discernible color change.

The present invention is further directed to such a method in which the fluid sample is combined with an aqueous solution to allow any ammonia in the fluid sample to dissolve into the aqueous solution. The aqueous solution is then contacted with a sensor which comprises a porous, hydrophobic polymer having a dye embedded within its pores. The dye has the capacity to be deprotonated and undergo a color change in the presence of ammonia gas. When the aqueous solution is contacted with the polymer under conditions which enable the pores of the polymer to be permeated by gaseous ammonia derived from the fluid sample, therefore, a color change in the dye is optically detected.

A further aspect of the invention is a device for carrying out the breath test. The device may be employed to detect the presence of a bacteria in the gastrointestinal tract of a subject associated with the catalytic breakdown of urea to carbon dioxide and ammonia. In general, the device comprises a breath sampler including a disposable breath handler and a detection unit. The breath handler is valved to permit inhalation there through which bypasses the detection unit, but exhalation is directed through the detection unit. The presence of ammonia in the exhalation is sensed by an ammonia sensing membrane in the detection unit. In one embodiment, the detection unit includes a container carrying liquid in which the membrane is submersed. In another embodiment, the breath sampler is constructed for inhibiting acquisition of ammonia from sources within the subject's mouth.

### BRIEF DESCRIPTION OF THE DRAWINGS

These and other features, aspects, and advantages of the present invention will become better understood with regard to the following description, appended claims and accompanying figures where:

FIG. 1 is an elevation of a breath sampler illustrated in use by a subject;

FIG. 2 is an elevation of the breath sampler of FIG. 1 with parts broken away to show internal construction;

FIG. 3 is a perspective of the breath sampler and an optical reader into which a portion of the breath sampler may be received;

FIG. 4 is an elevation of a breath sampler and optical reader of a second embodiment held by the subject in use;

FIG. 5 is the breath sampler of the second embodiment;

FIG. 6 is an exploded perspective of the breath sampler and optical reader of the second embodiment together with a battery charger;

FIG. 7 is a depiction of the total amount of ammonia and ammonium, NH4+3, detected by the sensors of the present invention versus the known amounts of ammonia and ammonium in the test solutions ranging from about 0.1 ppm to about 100 ppm as measured by a chemical analyzer (Cobas);

FIG. 8 is a kit of the present invention;

FIG. 9 is a breath sampling system including a breath sampler and optical reader of a third embodiment;

FIG. 10 is an exploded perspective of the breath sampling system of FIG. 9;

FIG. 11 is a top plan view of the breath sampler of FIG. 9 illustrating a second position of a breath handler of the breath sampler in phantom; and

FIG. 12 is a section of the breath sampler of FIG. 9.

Corresponding reference characters indicate corresponding parts throughout the several views of the drawings.

### DETAILED DESCRIPTION OF THE INVENTION

Among the various aspects of the present invention, is the provision of various methods and apparatus for use in the detection of H. pylori and other bacteria which are associated with the catalytic breakdown of urea to carbon dioxide and ammonia in the gastrointestinal tract. In general, the method comprises administering urea to a subject, obtaining a fluid sample from the subject after the administration of the urea, contacting the fluid sample with a sensor which undergoes a color change in the presence of ammonia, and optically reading the sensor to detect ammonia in the fluid sample.

Advantageously, the method of the present invention provides a means to detect the presence of any bacteria associated with the degradation of urea to ammonia and carbon dioxide when colonized in the gastrointestinal tract of a subject. In one embodiment, the subject is a mammal. In another embodiment, the subject is a livestock animal, zoo animal, a companion animal or a human. In a preferred embodiment, the subject is a human.

A number of bacterial strains possess urease associated activity and colonize within the gastrointestinal tract of a variety of subjects. For example, when the subject is a human, many species of Helicobacter, including H. pylori, H. heilmanii colonize within the gastrointestinal tract (Hirschl A. M., Wien Klin Worchenschr, 106(17): 538-42, 1994). Additionally, a coccoid organism, preliminarily suggested to be related to Staphylococcus, has been cultivated from gastric biopsy specimens obtained from human subjects (Solnick and Schauer, Clinical Microbiology Reviews, 14(1): 59-97, 2001). Moreover, when the subject is a primate, H. nemestinae has been show to colonize within the gastrointestinal tract (Solnick and Schauer, Clinical Microbiology Reviews, 14(1): 59-97, 2001). Further, when the subjects are felines or canines, many distinct species of Helicobacter including H. bizzozeronii, H. salomonis and H. felis colonize within the gastrointestinal tract (Solnick and Schauer, Clinical Microbiology Reviews, 14(1): 59-97, 2001).

The bacteria to be detected, accordingly, will vary greatly depending upon the particular subject being examined. In one embodiment, the subject is a human and the bacteria to be detected is a Helicobacter. In this embodiment, for example, the bacteria to be detected may be H. heilmanii or H. pylori. Currently, the detection of H. pylori in human subjects is of particular clinical importance.

H. pylori and other bacteria possessing urease associated activity that colonize in the gastrointestinal tract of a subject, as described above, have been shown to cause a number of gastrointestinal disorders. These disorders include gastritis, peptic ulceration, gastric cancer, non-ulcer dyspepsia, duodenal ulcers, gastric ulcers, duodenitis, gastric non-Hodgkin's lymphomas, intestinal metaplasia, adenocarcinoma, and esophagitis. Accordingly, another aspect of the present invention is a method to diagnose gastrointestinal disorders caused by the bacteria described above. The methods of the invention may be employed to detect any stage of bacterial infection including early or late stages. Preferably, however, diagnosis is made during the early stages of infection before significant damage is done to the gastrointestinal tract of the subject.

Bacteria having urease associated activity that colonize in the gastrointestinal tract of a subject, as described above, have also been linked to disorders associated with the liver. In particular, H. pylori and H. heilmanii have been associated with hepatitis (McCathey et al, Helicobacter, 4(4): 249-59, 1999). Accordingly, another aspect of the present invention is a method for the diagnosis of liver disorders caused by the bacteria described above. Preferably, diagnosis is made during the early stages of infection before significant damage is done to the liver tissue.

In each of these methods, it is preferred that substantially all substances present in the gastrointestinal tract of the subject that either may lead to the production of ammonia, such as a food source, or may impact the urease associated activity of the bacteria, are eliminated prior to administration of the urea source to the subject. Thus, for example, the subject preferably fasts for at least about 4 hours, typically about 4 to about 24 hours prior to administration of the source of urea and collection of the fluid sample. More preferably, the subject fasts for about 6 to about 12 hours prior to administration of the source of urea collection of the fluid sample.

Bacteria possessing urease associated activity may reside in the oral cavity. When the fluid sample to be obtained from a subject is a breath sample, therefore, the subject may optionally be given a mouth wash comprising an antibacterial agent prior to administration of the source of urea. The mouth wash will thus tend to decrease the population of such bacteria in the oral cavity and thereby reduce potential bias in the sample collection process.

In general, any source of urea, in solid or liquid form may be administered to the subject. Such sources include any composition that may be converted to urea in vivo or otherwise serve as a substrate for the urease associated activity of the bacteria to be detected. By way of example, the urea source may constitute urea, per se, or it may be a derivative of urea. In one embodiment, the urea source is carbonyldioxide (H2NCONH2) which is commercially available from a variety of sources such as Sigma-Aldrich (Saint Louis, Mo.).

The amount of the urea source administered to the subject is preferably sufficient to produce a detectable concentration of ammonia in a fluid sample taken from a subject, without undue adverse side effects to the subject, such as toxicity, irritation or allergic responses. For a particular subject, the amount may vary and generally depends upon a variety of factors such as the form of the urea source, the particular fluid sample to be used, the weight of the subject, and species of the subject. In general, however, the amount administered will be from about 1 milligram to about 20 milligrams of urea per kilogram body weight of the subject.

The urea source may be administered to the gastrointestinal tract of the subject by any generally known method. In one embodiment, administration is by oral ingestion of urea, in single or multiple doses. The particular dosage form used to administer the urea may be, for example, in solid tablets or capsules, or in liquid solutions or emulsions. Moreover, urea may be administered essentially in pure form, as detailed above, or as part of a composition. Compositions useful in administration of urea may also contain pharmaceutically-acceptable components such as, for example, diluents, emulsifiers, binders, lubricants, glydants, colorants, flavors and sweeteners. Suitable components included in the composition, however, preferably do not interfere with hydrolysis of the urea, or generate appreciable quantities of carbon dioxide or ammonia in the gastrointestinal tract. A preferred optional component is one which delays gastric emptying, thereby increasing the length of time that the administered urea is present in the stomach.

After administration of the urea source to the subject, a period of time sufficient for the bacteria to catalyze urea to ammonia and carbon dioxide is allowed to elapse before collection of the fluid sample. In one embodiment, about 1 to about 120 minutes elapse after administration of urea prior to collection of the fluid sample. In another embodiment, about 5 to about 60 minutes elapse after administration of urea prior to collection of the fluid sample. In still another embodiment, about 10 to about 30 minutes elapse after administration of urea prior to collection of the fluid sample.

After a suitable period has elapsed, a fluid sample is obtained from the subject. The fluid sample may be any fluid, gaseous or liquid, containing a detectable amount of ammonia gas resulting from the urease associated activity of the bacteria to be detected. Suitable fluid samples include a breath sample, a saliva sample, a perspiration vapor sample, a gastric reflux sample and a tear sample. The volume of the fluid sample collected will depend, in part, upon the fluid type and source and the sensitivity of the sensor employed and can readily be determined by a skilled artisan.

In one embodiment, the fluid sample is a breath sample obtained from the subject's lungs through the nose, mouth, trachea, or other external orifice of the subject. Typically, and most conveniently the breath sample may be collected by having the subject exhale (or blow) into a gas collection apparatus. For example, the subject may exhale into a balloon and the contents of the balloon may be directly or indirectly transferred to a sensor for analysis. Alternatively, and more preferably, the subject exhales directly into a sensor apparatus of the type depicted in any of the various figures appearing and described in greater detail elsewhere herein.

In another embodiment, the fluid sample is a saliva or tear sample. A saliva sample may be collected by any means generally known in the art. For example, the saliva sample may be collected by having the subject expectorate into the collection device. If the subject has difficulty doing this, substances may be contacted with the buccal cavity to generate a reflex stimulation of saliva by the saliva glands. These substances illustratively include citric acid or milk. Alternatively, the saliva or tear sample may be collected by a sample probe. The sample probe may comprise a swab on a support stick which is placed into the mouth of a subject to collect saliva or the eye of the subject to collect a tear which is then transferred from the swab to the collecting apparatus.

In still another embodiment, the fluid sample is a perspiration vapor sample. The perspiration vapor sample may be collected by any means generally known in the art. For example, the perspiration vapor sample may be collected using a dermal patch device which is placed directly on the skin of the subject. Under the influence of the subject's body heat, which is readily conducted from the surface of the skin through the liquid phase, the liquid water component of the perspiration will tend to evaporate. Such volatilized water can thereby pass through the gas permeable filter and leave the patch device. The device can further contain a microbead layer, where microbeads can desirably attach to the desired ammonia, thereby preventing it from escaping as a vapor through the gas permeable filter. In one embodiment, the trapped sample is then placed into the collecting apparatus and the presence of ammonia gas is then determined as described herein. In an alternative embodiment, the patch employed to collect the perspiration vapor sample has a dye embedded within its pores that changes color in response to the presence of ammonia gas. Accordingly, in this embodiment, the presence of ammonia gas in the perspiration vapor sample may be directly determined by observing a color change of the dye in the patch.

In yet another embodiment, the fluid sample is a gastric reflux sample. The gastric reflux sample may be collected by any means generally known in the art. For example, the gastric reflux sample may be collected by stimulating the throat or larynx producing vomiting-reflexive belching, called “eructation.” A round-shaped structure at its tip can be used to stimulate the throat or larynx, preventing the inside of the oral cavity from scratching. The gastric gas is thereby directed to the oral cavity and into the measuring device.

Regardless of the nature of the fluid sample or the means for its collection, a sensor is used to detect the presence of ammonia in the fluid sample. In one embodiment, the fluid sample (whether gas or liquid) is directly contacted with the sensor. In another embodiment, the fluid sample (whether gas or liquid) is first combined with an aqueous solution to allow ammonia in the fluid sample to dissolve into the aqueous solution and the aqueous solution is then contacted with the sensor.

In those embodiments in which the fluid sample is first combined with an aqueous solution, the aqueous solution, in theory, may contain any composition which does not interfere with the detection of ammonia. For example, the aqueous solution preferably does not contain any compositions which react or otherwise deleteriously interact with ammonia (or ammonium) in the fluid sample. By way of illustration, the aqueous solution preferably does not contain compounds that covalently bind to or that form complexes with ammonia. In one embodiment, the aqueous solution comprises sterile water which has been adjusted to a desired pH. Ammonia (or ammonium) present in the fluid sample will readily dissolve in such a solution and, as described elsewhere, the ammonia can thereafter be detected using an ammonia sensor.

In an aqueous solution, ammonia may exist in an ionic (NH4+) or non-ionic (NH3) form and the pH of the solution dictates which of these forms is predominant. For example, NH4+ is predominant in acidic solutions while NH3 is predominant in basic solutions. Because the sensor of the present invention is tuned to measure ammonia, however, it is preferred that the aqueous solution have a neutral or basic pH. In one embodiment, the aqueous solution has a pH of 7 to about 9.5. In another embodiment, the aqueous solution has a pH from about 8.0 to about 9.0. Any suitable compound which does not interfere with the assay may be added to the aqueous solution in order to adjust the pH to a desired value. Exemplary compounds which may be used for this purpose include the hydroxides of alkali metals and alkaline earth metals, such as sodium hydroxide or potassium hydroxide.

To detect the presence of ammonia in the fluid sample, the fluid sample itself or an aqueous solution which has been combined or otherwise contacted with the fluid sample is brought into contact with an ammonia sensor. In general, the sensor is a polymer carrying a dye which is capable of being deprotonated and undergoing a color change in the presence of ammonia. When the sensor comes into contact with ammonia, therefore, the dye undergoes an optically detectable color change which can be observed by a visual or other optical inspection of the sensor.

In one embodiment, the sensor comprises a porous, hydrophobic polymeric material and the dye is embedded within the pores but is substantially absent from the remainder of the exposed surface of the material. After a fluid sample, e.g., a breath sample, is combined or otherwise contacted with an aqueous solution for a period sufficient for ammonia (ammonium ions) to dissolve into the solution, the aqueous solution is brought into contact with the sensor. Because the dye is substantially absent from the surface of the sensor, the exposed surface of the sensor will not undergo a significant color change. Ammonia dissolved in the aqueous solution, however, can permeate the pores of the sensor and deprotonate the dye to effect a discernible color change. Significantly, the pore size and hydrophobic character of the sensor combine to effectively exclude liquid from the pores of the sensor. For all practical purposes, therefore, the pores of the sensor are impermeable to the aqueous solution, and as a result, the sensor will undergo a discernible color change in response to the presence of ammonia (ammonium ions) in the fluid sample but not in response to the pH of the aqueous solution. Stated another way, the sensor does not respond to the pH of the fluid sample but rather, to the presence of ammonia in the fluid sample independent of the pH of the fluid sample. Any significant color change by the sensor, therefore, is a positive indication that the pores have been permeated by ammonia.

In general, the degree of hydrophilicity or hydrophobicity can be determined by reference to the contact angle of a droplet of water placed on the surface of the polymer. As used herein, a polymer is considered to be hydrophilic if the contact angle is less than 30 degrees; conversely, a surface is considered to be hydrophobic if the contact angle of a drop of water placed on the surface is greater than about 100 degrees. (M. Cheryan, Ultrafiltration and Microfiltration Handbook 245-46 Technomic Publishing Co.). More preferably, the a surface is considered to be hydrophobic if the contact angle is between 100 and 150 degrees. Even more preferably, a surface is considered to be hydrophobic if the contact angle is 110 degrees.

As previously mentioned, the hydrophobicity of the sensor material, combined with the porosity of the sensor material can be controlled to substantially prevent aqueous solutions from permeating the pores of the sensor material. In one embodiment, the pore size is about 9.0 micrometers or less, preferably from about 3.5 microns to about 0.2 microns, more preferably about 2.5 microns or less as determined by bubble point pressure definition. In another embodiment, the average pore size of the polymer can range from about 2.5 microns to about 1 micron, preferably from about 2.0 microns to about 1.6 microns. Of course, it will be apparent to those skilled in the art that it is possible, and perhaps desirable, that the polymers can be made to include any variety of different and suitable pore sizes.

In an alternative embodiment when the fluid sample is a vapor, such as a breath sample, the fluid sample may be directly contacted with a hydrophobic polymer. Because the sensor is not coming into contact with a liquid solution in this embodiment, there is a reduced risk that a species other than ammonia gas is responsible for a color change in the sensor. Accordingly, the dye may be, but need not be contained substantially exclusively in the pores of the sensor material (i.e., the dye may be carried on the remaining exposed surface of the sensor) in this embodiment.

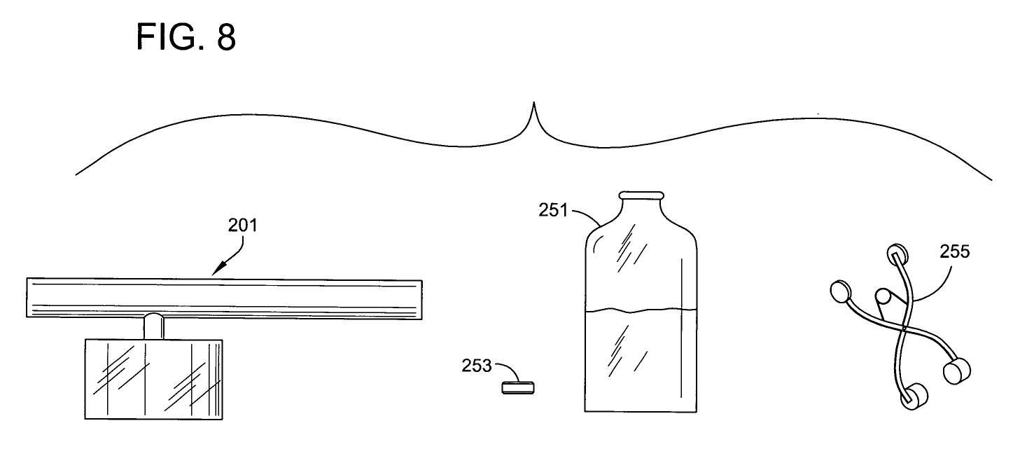

In accordance with the method of the present invention, a color change in the sensor reflects the presence of ammonia and not merely the pH of an aqueous solution which is in contact with the sensor. This color change is detected optically. In one embodiment, the color change is detected visually by the observation of the subject or a technician assisting the subject in the assay. In this embodiment, the color change may merely be read to confirm the presence or absence (but not the concentration) of ammonia in the fluid sample; alternatively, the degree of color change may be used as a quantitative or semi-quantitative measure of the concentration of ammonia in the fluid sample. In another embodiment, an optical reader is used to monitor the color change; for example, the optical reader may be a calorimetric reader, such as a spectrophotometer or laser, as more completely described in U.S. patent application Ser. No. 10/024,170 entitled “Ammonia and Ammonium Sensors,” the entire content of which is hereby incorporated by reference (FIG. 8). The color change may merely be read by the optical reader to confirm the presence or absence (but not the concentration) of ammonia in the fluid sample; alternatively, the degree of color change may be used as a quantitative or semi-quantitative measure of the concentration of ammonia in the fluid sample.

In one embodiment, the color change is reversible. In this manner, the presence (or amount) of ammonia in a fluid sample or aqueous solution may be optically detected by observing a color change in the dye as a function of time.

The sensor may comprise any of a variety of polymeric materials. For example, the sensor may consist of polypropylene, polytetrafluoroethylene (“PTFE”), polyvinylidene difluoride (“PVDF”), fluorinated ethylene propylene polymers (“FEP”), acrylic-based polymeric compounds, acrylic-based fluorinate polymers, polycarbonate, polypropylene, polyvanilidine chloride, dimethyl polysiloxane and copolymers thereof, or combinations thereof.

The sensor may also assume any of a variety of geometric shapes. For example, the sensor may comprise regular or irregularly shaped particles, e.g., beads, relatively thin layers (supported or unsupported by other materials), or any of a wide variety of shapes which may be useful for optical readers or mere observation. In one embodiment, the polymer is in the form of a membrane.

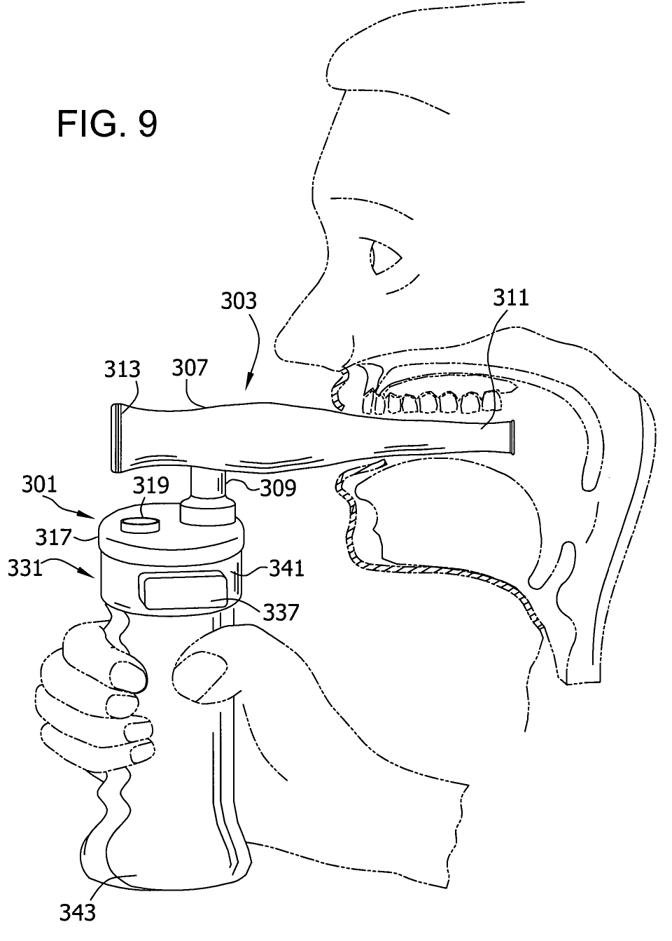

In one embodiment, the dye is intimately embedded or bound within the porous structure of the polymer such that a negligible amount, if any, dye leaches from the polymer when the polymer is exposed to the aqueous solution. In general, any method known in the art may be employed to embed or bind the dye to the pores of the polymer, including those methods described in U.S. patent application Ser. No. 10/024,670 entitled “Hydrophobic Ammonia Sensing Membrane,” which is hereby incorporated by reference in its entirety (FIG. 9).

In one embodiment of the present invention, the dye is associated with the polymer in a casting process. During casting, the polymer and the dye are formed into a casting solution containing a suitable solvent. Any suitable amounts of the polymer and dye can be blended or mixed into the casting solution. Typically, the casting solution includes at least about 0.1% by weight of the dye in a solution containing about 14% to about 24% by weight of polymer, and more preferably about 19% to about 21% by weight of polymer. The casting solution is then poured onto substrate, such as a mesh material, or glass substrate, and further processed by immersing the casting solution in an acidic solution under suitable conditions such that a precipitate is formed. In an embodiment, the acidic solution used during precipitation contains a suitable amount of methanol, including about 50% to about 100% by weight of methanol, preferably about 90% or more by weight, and has a pH of about 3 to about 4, and more preferably, about 3. The precipitate is then further washed and dried under suitable conditions to form the polymer with the dye embedded within the pores of the polymer. In an embodiment, the processed polymer can be dried at temperature ranging from about room temperature to about 100° C., and preferably, at about 60° C. An example of the casting procedure illustrative of an embodiment of the present invention is detailed below in Example 1.

In another method, the dye is associated with the polymer by dip coating. During dip coating, a polymer preformed to the desired shape, such as a membrane, is immersed in an aqueous coating solution containing a dye and a solvent. In an embodiment, the solvent includes isopropyl alcohol, acetone, mixtures thereof and other suitable solvent materials. The coating solution can include any suitable amount of the dye and the solvent. In an embodiment, the coating solution includes at least about 0.05% by weight of the dye, more preferably, at least 0.2% by weight of the dye, in an aqueous solution containing about 10% to about 50%, and more preferably, about 30% by volume of the solvent. An example of the dip coating process illustrative of an embodiment of the present invention is discussed below in Example 2.

Any dye that is sensitive to and responds to changes in the amount of ammonia that permeates the pores of the polymer may be employed. In a preferred embodiment, the dye is a pH sensitive dye that becomes deprotonated and undergoes a color change in the presence of ammonia gas. Suitable pH sensitive dyes include bromophenol blue, bromothymol blue, methyl yellow, methyl orange, 2,4-dinitrophenol, 2,6-dinitrophenol, phenol red, cresol red and any mixtures thereof. Preferably, the dye selected is capable of reverting back to its original color when not contacted by ammonia gas. In addition, the dye selected is also capable of imparting to the sensor the ability to undergo a degree of color change which is directly proportional to the amount of ammonia gas that permeates into the polymer.

In one embodiment of the invention, a fluid sample is taken from the subject prior to administration of the urea source and the ammonia concentration is determined in accordance with the steps detailed above. The subject is then administered a urea source, as detailed above, and a number of fluid samples are then collected from the subject. The presence or concentration of ammonia gas in each sample is independently determined. Typically, about 1 to about 20 fluid samples are collected and quantified. Even more preferably, about 5 to about 10 fluid samples are collected.

The collection and testing of a fluid sample prior to the administration of urea, and one to several fluid samples after the administration of urea, is particularly advantageous to increase the accuracy of the method. By way of illustration, for example, prior to administration of urea, only a nominal amount of ammonia gas will be present in the fluid sample, e.g. on the order of less than about 1 part per million. After the administration of urea, however, the amount of ammonia gas present in the fluid sample will increase by a magnitude of about 10 to about 1000 fold. Accordingly, by comparing the amount of ammonia gas present in both pre and post urea fluid samples, the method of the present invention provides an extremely accurate means to determine the presence of a bacteria possessing urease associated activity in the gastrointestinal tract of a subject.

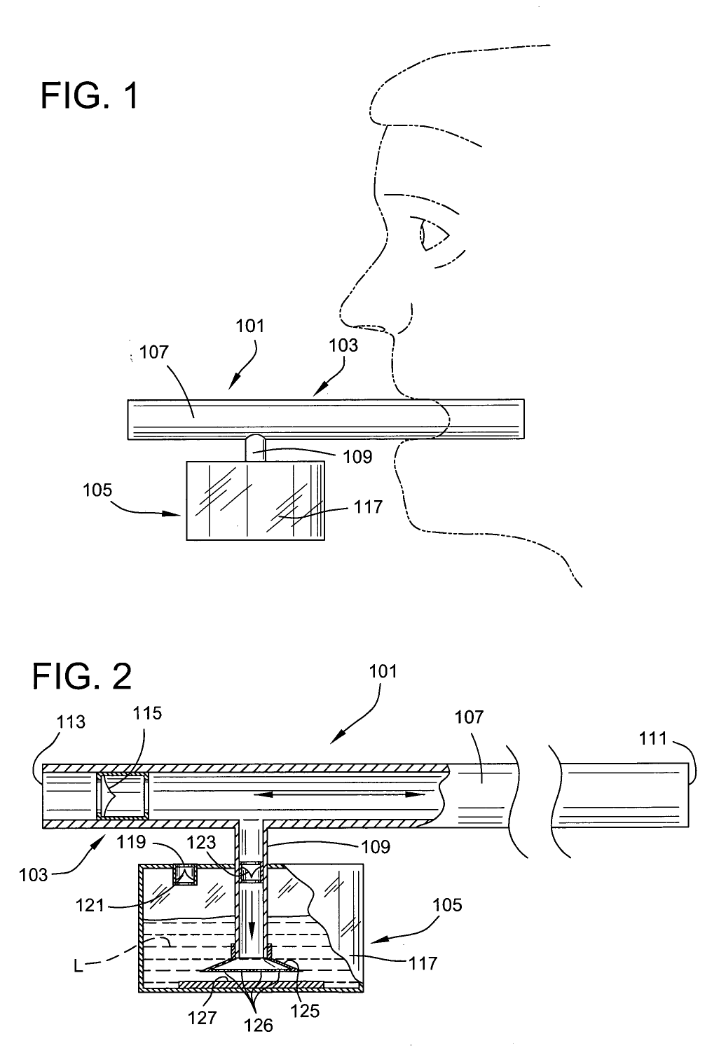

Although the method of the present invention may be carried out in a variety of apparatus, it is preferably carried out in a first embodiment using a breath sampler, generally indicated at 101 in FIG. 1. The breath sampler is shown in FIG. 1 in use by a subject to obtain a breath sample for indication of the presence of H. pylori infection in the subject's gastrointestinal tract. The breath sampler 101 comprises a breath handler and a detection unit, indicated in their entireties by reference numerals 103 and 105, respectively. The detection unit 105 is connected to the breath handler 103 for receiving the subject's breath sample, which may consist of one, but is usually several exhaled breaths. Generally, the breath sampler 101 is configured to permit inhalation through the breath handler 103 and exhalation through the detection unit 105. The breath handler 103 includes an elongate tube 107 and a sample collection branch 109 formed in the illustrated embodiment as one piece with the breath handler from a suitable material. Referring now also to FIG. 2, the tube 107 is open at both a mouthpiece end 111 and an intake end 113 so that air may pass through the tube from the intake end through the open mouthpiece end. A check valve 115 in near the intake end 113 of the tube 107 restricts flow through the tube at the intake end to a direction toward the mouthpiece end 111 and blocks flow out of the intake end. In other words, the check valve 115 permits air to be taken in through the intake end 113, but does not permit the subject's breath to pass out through the intake end. The check valve 115 may be of any suitable construction, such as a generally cone-shaped diaphragm having a slit in its small end. Air passing in from the intake end 113 passes through a larger base of the diaphragm and forces open the slit to pass through the check valve 115. However, the subject's exhalation bears against the generally conical walls of the exterior of the diaphragm of the check valve 115 and pushes the walls inward toward the center, holding the slit in a closed position so that the flow of exhalation out through the intake end 113 is blocked.

The breath sample from the subject is directed downward into the sample collection branch 109 and into a container 117 of the detection unit 105. The container 117 has a vent 119 which permits excess gas in the container to be exhausted to the atmosphere so that exhalation may flow into the container. A check valve 121 in the vent 119 permits flow of air out of the container 117 upon exhalation, but when the subject is not providing air pressure, closes the container vent to the influx of ambient air and to outflow of liquid L. Another check valve 123 disposed in the collection branch 109 permits the breath sample to flow through the branch and into the container 117, but inhibits withdrawal of gas or liquid L from the container upon inhalation. Moreover, the check valve 123 helps to keep the liquid L from spilling out of the container 117. The construction and operation of the check valves 121, 123 can be the same as check valve 115 described above, except that the orientation of the check valves 121, 123 is reversed to permit the breath sample to flow outwardly from the mouthpiece end 111 through the tube 107, sample collection branch 109, container 117 and vent 119, and to block air flow through the vent, sample collection branch, container and tube toward the mouthpiece end. The check valves 115, 121, 124 are desirably biased to a closed position in the absence of a pressure differential across the valve so that the container 117 is normally substantially isolated from the breath handler 103 and the ambient air. It is to be understood that while the check valves 115, 121 provide certain advantages and conveniences in use, they may be omitted without departing from the scope of the present invention. For example, the intake end of the tube may be closed off (not shown). Generally speaking, the breath handler 103, detection unit 105, container 117 and check valves 115, 121, 123 are made of a medical grade plastic capable of being initially sterilized. However, the breath sampler 101 is preferably disposable so that an inexpensive plastic is desirably employed.

In the embodiment of FIG. 1, the container 117 of the detection unit 105 is constructed for holding a volume of liquid L, such as sterilized water, and the lower end of the sample connection branch 109 which includes a diffuser head 125 is immersed in the liquid. Exhalation passes out of the sample collection branch 109, through small openings 126 in the diffuser head 125 and into the liquid L. Diffusion of the breath sample by the head 125 facilitates retention of any ammonia in the sample by the liquid L. An ammonia sensing membrane 127 is located on the bottom of the container 117 in opposed relation with the diffuser head 125 so that the breath sample leaving the collection branch is spread within the liquid L and over the membrane. The membrane 127 is of the type which detects the presence of ammonia (and ammonium) in the breath sample and indicates the presence of ammonia through a change of color. Examples of suitable ammonia sensing membranes are described in co-assigned U.S. patent application Ser. No. 10/024,170, entitled Ammonia and Ammonium Sensors, and U.S. patent application Ser. No. 10/024,670, entitled Hydrophobic Ammonia Sensing Membrane, previously incorporated herein by reference. The membrane 127 is attached in a suitable manner, such as by ultrasonic or thermal welding to a bottom wall of the container 117.

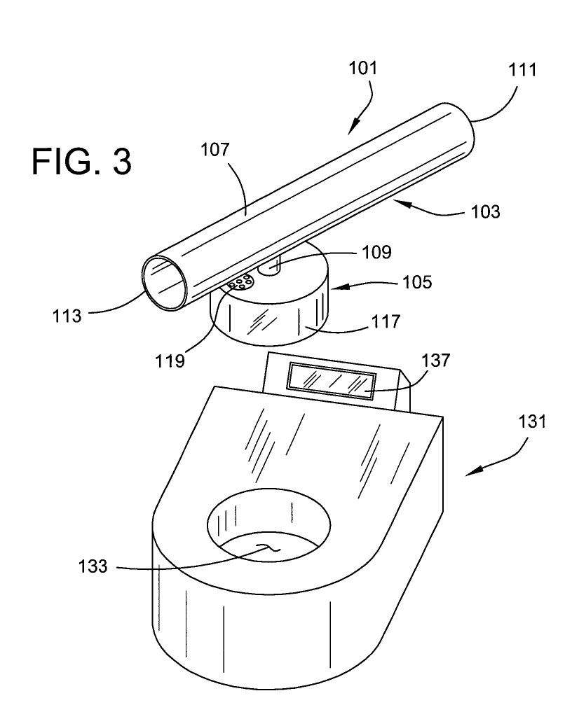

An optical reader, indicated generally at 131 in FIG. 3, has an opening 133 in an upper surface for receiving at least the lower end of the container 117. In the illustrated embodiment, the container 117 is formed of an optically clear material so that the membrane 127 can be examined by the optical reader 131 through a wall of the container. The optical reader 131 may be of the type which sends light signals toward the membrane 127 so that a photo-detector (not shown) may read the color of the membrane. The optical reader 131 has a display 137 to output a suitable message indicative of the presence or absence of ammonia in the sampled breath based on the color of the membrane 127 detected. A suitable optical reader is disclosed in the aforementioned U.S. patent application Ser. Nos. 10/024,170 and 10/024,670.

In use, the subject places the mouthpiece end 111 of the breath handler tube 107 in the mouth and seals around the tube with the lips. Typically, the nasal passages are occluded, such as by placing a clip (not shown) on the nose, so that the subject breathes only through the breath sampler 101. Previously, the subject will have blown a baseline reading through the breath sampler 101 which is read by the optical reader 131. The subject will have subsequently been prepared and have ingested urea so that ammonia may be generated in the presence of H. pylori in the gastrointestinal tract. The subject then draws air into the lungs by inhaling. The check valves 121, 123 prevent liquid L or any gas from the container 117 from being aspirated into the subject's lungs by blocking flow toward the mouthpiece end 111. However, air passes freely into the intake end 113 and through the check valve 115. The subject then exhales, and breath passes into the tube 107 through the mouthpiece end 111 where it is blocked by the check valve 115 at the intake end 113, but may pass through the check valve 123 in the sample collection branch 109 into the container 117 and thence out of the container through the check valve 121 and vent 119. The breath sample passes into the liquid L and over the ammonia sensing membrane 127 which changes color if ammonia (or ammonium) is present in a sufficient quantity in the breath sample. Preferably, the subject should breathe in and out several times to provide sufficiently large sample to the ammonia sensing membrane 127. The particular construction of the breath sampler 101 shown and described herein makes it much more convenient for the subject to inhale and exhale multiple times without unsealing the lips from the breath handler tube 107 or aspirating liquid from the container 117.

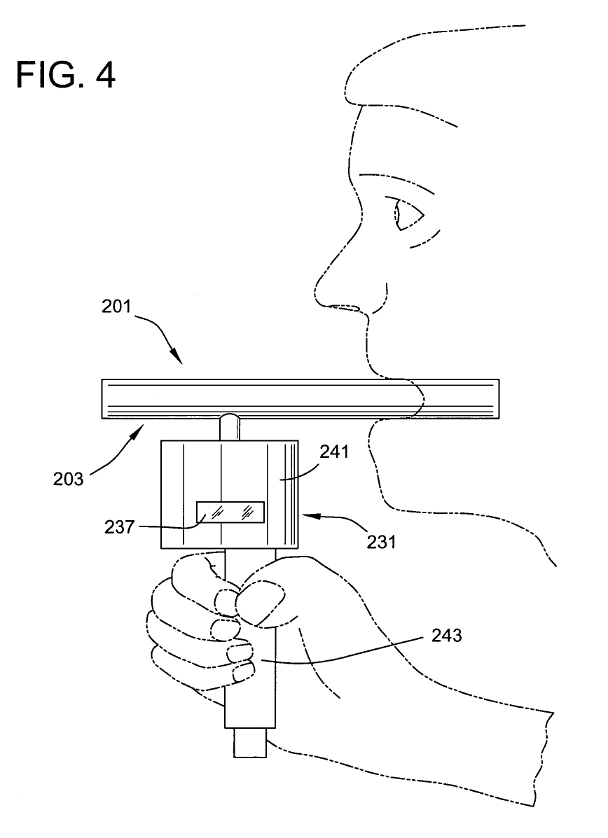

Referring now to FIGS. 4-6, a breath sampler and optical reader of a second embodiment are generally designated at 201 and 231, respectively (collectively, “a breath sampling system”). The breath sampler 201 comprises a breath handler 203 and a detection unit 205. Corresponding parts of the breath sampler 201 and optical reader 231 will be indicated by the same reference numerals as the breath sampler 101 and optical reader 131 of the first embodiment, plus 100. The construction of the breath handler 203 may be substantially the same as the breath handler 103 of the first embodiment. However, the detection unit 205 is a much flatter container 217 defining a shallow internal volume through which air passes to a vent 219 and the container does not hold any liquid. A check valve 221 is located at the vent 219 and a check valve 223 is located in the collection branch 209, as with the first embodiment. The diffuser head 225 has its lower surface disposed just above the ammonia sensing membrane 227 so that the breath sample passes out of dispersed openings 226 and is spread over and contacts the membrane. In this embodiment, the ammonia sensing membrane 227 is operable to detect ammonia directly from the breath sample without passage into a liquid.

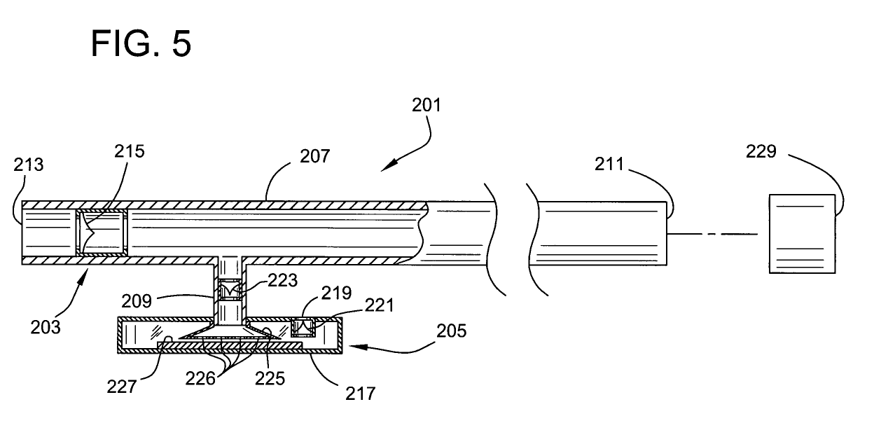

It is to be understood that liquid may be used in the container 217, or for that matter not used in the container 117 of the first embodiment, without departing from the scope of the present invention. Where liquid is employed, the container is likely, but not necessarily larger in volume. Liquid has an advantage in that it accumulates ammonia in multiple exhalations from the subject, making it easier for the ammonia sensing membrane (127, 227) to detect the minute amounts of ammonia in the breath sample. If liquid is not used, as in the embodiment illustrated in FIGS. 4-6, it may be desirable to provide a cap 229 (FIG. 5) which can be fitted over the mouthpiece end 211 of the breath handler 203 to assist sealing the sample within the breath sampler 201. It is envisioned that the cap 229 might be used instead of the check valve 223 in a more inexpensive version of the breath sampler 201, or omitted when the check valve 223 is present.

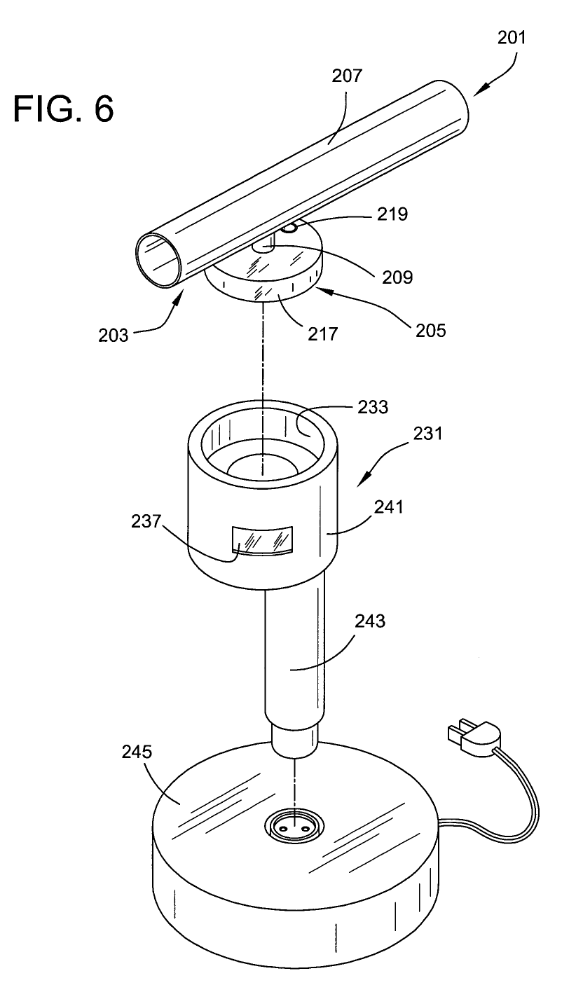

The smaller, portable optical reader 231 includes housing having a reader portion 241, an output display 237, and a handle 243 which houses a rechargeable battery (not shown). The battery allows the optical reader 231 to be self-contained, i.e., operable to provide a reading remote from any other power source. The breath sampler 201 is preferably disposable and constructed for releasable, snap together attachment to the optical reader 231. However, other forms of attachment of the breath sampler 201 to the optical reader 231 may be employed. The optical reader 231 is light weight and portable so that it can be attached to the breath sampler 201 as it is being used by the subject for an immediate indication of an H. pylori infection in the gastrointestinal tract. As shown in FIG. 4, the subject may hold the handle 243 of the optical reader 231 while breathing through the breath sampler 201 mounted on the optical reader. A battery charger 245 is provided for recharging the battery housed in the handle 243 of the optical reader 231 (FIG. 6).

A further aspect of the invention provides a kit to detect the presence of a bacteria capable of catalyzing urea to carbon dioxide and ammonia. The kit comprises a sterile disposable breath sampler (e.g., breath sampler 201), a bottle 251 which contains urea concentrate, which can have a different volume with variant concentrations of urea, or a tablet 253 with equivalent solid urea (USP), and a nasal cannula 255.

In one embodiment, the subject will drink the urea concentrate from the bottle 251 (or take the tablet 253) and then apply the nasal cannula 255 to occlude the nasal passages. After the appropriate amount of time has gone by, the subject breathes through the breath sampler 201 for a breath ammonia measurement at designated time intervals. The optical reader 231 will display the measured ammonia with a present mathematical model to diagnose if the subject has been infected by bacteria that can catalyze urea into ammonia and carbon dioxide.

In one embodiment, the subject will drink the urea concentrate and then put on the nasal cannula to breath through the breath sampler for a breath ammonia measurement at designated time intervals. The optical reader will display the measured ammonia with a present mathematical model to diagnose if the subject has been infected by bacteria that can catalyze urea into ammonia and carbon dioxide.

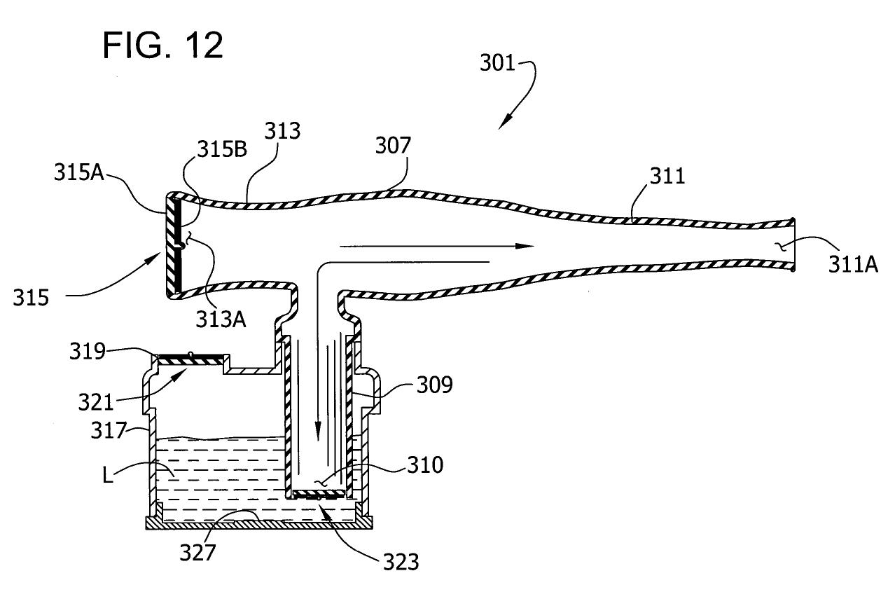

A breath sampler and optical reader, generally designated at 301 and 331, respectively, are shown in FIGS. 9-12. The breath sampler 301 comprises a breath handler 303 and a detection unit 305. Corresponding parts of the breath sampler 301 and optical reader 331 will be indicated by the same reference numerals as breath sampler 101 and optical reader 131 of the first embodiment, plus 200. The breath handler 301 has a construction similar to that of the breath handler 101, including a tube 307 and a sample collection branch 309. The tube 307 has a mouthpiece end portion 311 and an intake end portion 313 so that air may pass through the tube from an air intake 313A to a breath sample opening 311A in the distal end of the mouthpiece end portion (see FIG. 12). A check valve 315 located in the air intake 313A of the tube 307 restricts flow through the tube at the air intake end portion 313 to a direction toward the mouthpiece end portion 311 and blocks flow out of the tube through the air intake. The check valve 315 may be of any suitable construction, and is illustrated in FIGS. 9-12 as a disk including a rigid, perforated substrate 315A and a flexible diaphragm 315B having a central opening. Upon inhalation, the pressure drop behind the diaphragm 315B pulls it off of the substrate 315A, allowing air to flow in through the holes in the substrate and through the central opening of the diaphragm. Upon exhalation, positive air pressure behind the diaphragm 315B (and the resiliency of the diaphragm) urges the diaphragm against the substrate, covering the holes in the substrate and central hole in the diaphragm to block the flow of air past the check valve 315 out of the tube 307. A check valve 321 is located at a vent 319 in a collection container 317 of the detection unit 305, and a check valve 323 is located in the collection branch 309, as with the first embodiment. These check valves (321, 323) are of the same construction as the check valve 315 and are oriented to permit the flow of air as described above for the breath sampler 101 of the first embodiment. A greater or lesser number of check valves (e.g., valves 315, 321, 323) may be used without departing from the scope of the present invention. For example and without limitation, the check valve 321 at the vent 319 might be omitted. A lower end of the collection branch 309 includes a breath sample outlet 310 which opens into liquid L covering the membrane 327 (FIG. 12). It will be understood that the breath sampler 301 may operate without the liquid L and not depart from the scope of the present invention.

The mouthpiece portion 311 of the breath handler 303 of the third embodiment is tapered in both a width and height dimension toward the breath sample opening 311A at the end of the mouthpiece portion. It will be understood that the tapering may occur in only one of the dimensions without departing from the scope of the present invention. The tapering facilitates reception of the mouthpiece portion 311 into the mouth (see FIG. 9). Moreover, the length of the mouthpiece portion 311 is selected so that the breath sample opening 311A may be placed far back in the mouth, essentially at the throat. This placement tends to avoid exhaled air passing into the mouth significantly before it passes into the breath sample opening 311A. It is possible for the mouth to contain substances which can generate ammonia, which could give a false reading. The configuration of the breath handler 303 helps the breath sampler 301 to avoid collection of ammonia from this source.

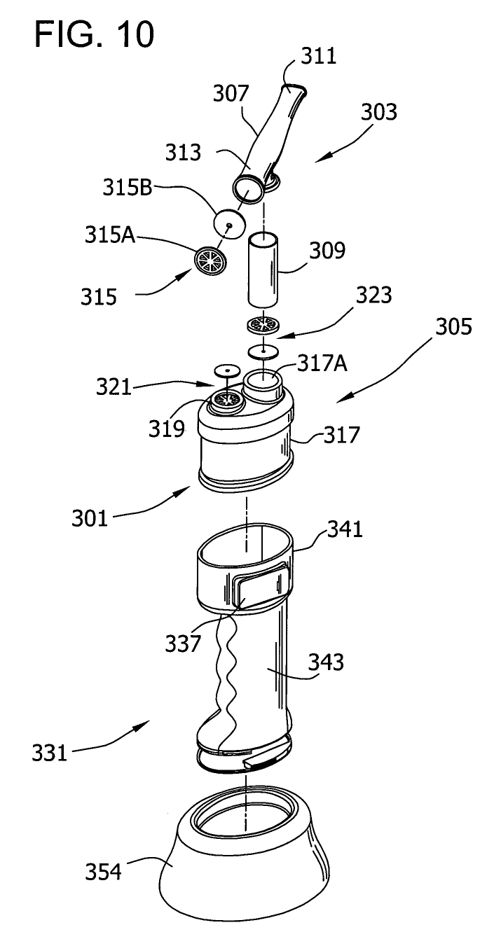

An optical reader 331 includes a housing having a reader portion 341, an output display 337, and a handle 343 which encloses a rechargeable battery (not shown). The battery allows the optical reader 331 to be self-contained, so that the subject may use the breath sampling system (breath sampler 301 and optical reader 331) apart from any fixed power source, as shown in FIG. 9. The subject holds the handle 343 of the optical reacher 331 and uses the optical reader to position the breath sampler 301 for taking the mouthpiece portion 311 into the mouth. The breath sampler 301 is preferably disposable and capable of releasable snap-together connection with the optical reader 331, but other configurations are possible within the scope of the present invention. As with the breath sampling system of the second embodiment, the subject may receive an immediate indication from the portable optical reader 331 of the presence or absence of H. pylori in the gastrointestinal tract when the test is conducted. A battery charger 345 is provided for recharging the battery housed in the handle 343 (FIG. 10).

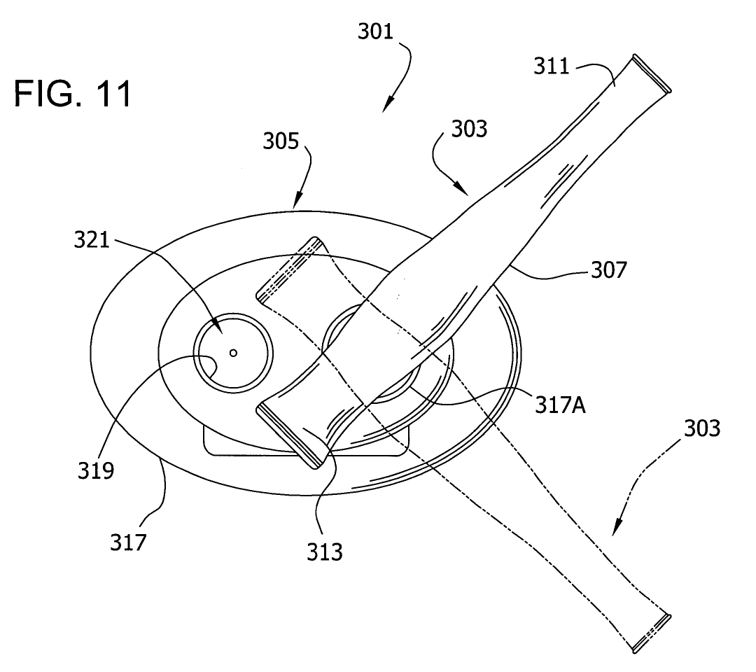

The collection branch 309 is received in the collection container 317 through an opening 317A and is pivotable in the opening with respect to the collection container about the axis of the collection branch. It is preferable to have the air intake 313A of the breath handler 303 offset from (i.e., not located directly above) the vent 319 so that as the subject breathes in and out several times through the breath handler 303, the subject does not inhale the material expelled from the collection container 317 through the vent. FIG. 11 illustrates two positions of the breath handler 303 with respect to the collection container 317. These two positions may be achieved by turning the breath handler 303 within the collection container 317. Thus, when the subject grasps the handle 343 of the optical reader 331 with either hand, the breath handler 303 may be turned relative to the collection container 317 to offset the air intake 313A of the breath handler from the vent 319 while allowing the optical reader to be held in a comfortable position for receiving the mouthpiece end portion 311 into the mouth. It is to be understood that the offset may be achieved in other ways (e.g., without pivoting of the breath handler) without departing from the scope of the present invention.

The detailed description set-forth above is provided to aid those skilled in the art in practicing the present invention. Even so, this detailed description should not be construed to unduly limit the present invention as modifications and variation in the embodiments discussed herein can be made by those of ordinary skill in the art without departing from the spirit or scope of the present inventive discovery.

All publications, patents, patent applications and other references cited in this application are herein incorporated by reference in their entirety as if each individual publication, patent, patent application or other reference were specifically and individually indicated to be incorporated by reference.

Without further elaboration, it is believed that one skilled in the art can, using the preceding description, utilize the present invention to its fullest extent. The following preferred specific embodiments are, therefore, to be construed as merely illustrative, and not imitative of the remainder of the disclosure in any way whatsoever.

### EXAMPLES

### Example 1

Construction of the polymer with a dye embedded in the pores by casting

A casting solution was prepared by blending a PVDF solution (e.g., hydrophobic membrane material) with bromophenol blue (pH sensitive dye). The casting solution included about 0.1% by weight of bromophenol blue in a solution containing about 20% by weight of PVDF. The PVDF solution was made with a suitable solvent including, for example, dimethyl acetamide, dimethyl formamide, triethyl phosphate, dimethyl sulfoxide or the like.

A methanol bath solution was prepared by mixing methanol with varying amounts of 0.1 N hydrochloric acid (“HCl”) to adjust the acid bath solution. The pH of the methanol bath solution ranged from about 3 to about 4. An acid washing solution was separately prepared with 0.1 N HCl having a pH of about 3.

A polyester support mesh passes through the casting solution continued in a V-shape dispensing device, sometime referred to as a V-box. The mesh exits and draws the casting solution through a slit on the V-box. The entire mesh structure becomes coated by and impregnated with the solution such that the casting solution is evenly spread on both sides of the mesh. The mesh substrate was a commercially available mesh material. Once exited, the casting solution was immersed in the methanol bath for about 3 minutes to about 18 minutes such that a precipitate formed. The precipitate was subsequently dried in air at about room temperature to about 80° C.

It should be appreciated that the above casting procedure can be suitably modified. In this regard, the dye can be added at any suitable stage during the casting procedure. For example, the dye can be added to the acid bath solution and then to the casting solution to form the precipitate. The casting procedure can also include additional washing of the precipitate prior to drying. The washing can be conducted with the acid wash solution as discussed above or other suitable washing media including water. Further, the drying stage can be conducted at room temperature or conducted under suitably higher temperatures in order to decrease the drying time. In addition, a variety of other suitable substrates in place of the mesh material can be utilized, such a glass substrate or a metal substrate.

### Example 2

Construction of the polymer with a dye embedded in the pores by dip coating

A dip coating solution was prepared by mixing about 0.2% by weight bromophenol blue into an aqueous solution containing about 30% by volume of isopropyl alcohol.

A polymer was prepared that included about 19% to about 21% by weight of PVDF. The PVDF was immersed into the dip coating solution for about 2 to about 8 minutes and then air dried to form the ammonia sensing polymer. It should be appreciated that the dip coating process of the present invention can be modified in a variety of different and suitable ways.

### Example 3

Physical Characteristics of the Sensors

A number of experiments were conducted to demonstrate the efficacy of the sensors of the present invention for detecting the presence of ammonia in a fluid sample. The sensors that were tested were prepared by procedures in accordance with an embodiment of the present invention as described above.

Porous Microstructure

The porous microstructure of the sensors made in accordance with an embodiment of the present invention were characterized employing a scanning electron microscopy technique. The test results demonstrated that the sensors of the present invention have a microporous sponge type structure with multilayered passageways in the mesh forming the pores. Dye is deposited on the internal surfaces of the mesh-like layers of the membrane.

pH Effects

The sensors of the present invention were tested to determine the effects of pH on the sensor's ability to detect ammonia. In an experiment, the sensors made in accordance with an embodiment of the present invention were placed in a 50 ml beaker containing 10 ml of distilled water and having a pH of about 7. The sensor, bromophenol blue, displayed no color change. After three minutes, an acid buffer solution, comprised of 15% sodium citrate, 15% citric acid, and 70% water was added to the beaker until the pH was about 4.7. Again, the sensor displayed no color change. Lastly, 0.1M NaOH was gradually added until the pH of the solution was about 10. Again, the sensor displayed no color change.

This demonstrates that the sensor will not react with changes of pH from ranges of pH 4.7 to pH 10. Therefore, changes in the pH have negligible, if any, effects of the calorimetric reactivity of the ammonia sensor of the present invention without the presence of ammonia.

Ammonia Selectivity

The ammonia sensors made in accordance with the present invention were tested to demonstrate the detection capabilities with respect to the detection selectivity for ammonia in comparison to other materials. In this test example, the sensors, bromophenol blue, were tested to evaluate their detection selectivity with respect to ammonia in the presence of carbon dioxide or ammonium.

The ammonia sensor was placed into a 50 ml small beaker. 10 ml of 1000 ppm CO₂ solution was then added into the beaker. The sensor displayed no visible color change. After three minutes, the sensor still displayed no color change. An acid buffer solution comprising of 15% sodium citrate, 15% citric acid, and 70% water was added until the pH was about 4.7. The acid buffer solution created CO₂. Again, there was no color change. Next, one drop of 1000 ppm NH₄OH was added to the beaker; allowing NH4+ to be present in the beaker, but not NH3. The sensor displayed no color change. Further, 0.1M NaOH was gradually added to the beaker to increase the pH of the solution to 10. Gradually, the sensor's color changed from the original yellow to light blue, blue, dark blue, and then darker blue as more NH4+was converted to NH3. Finally, to show that the sensor color is reversible, an acid buffer solution described above was again added to lower the pH. The sensor color visibly changed back to the original yellow.

The test results indicated that the presence of carbon dioxide had negligible, if any, effects on the detection of ammonia. In this regard, the sensor did not detect the carbon dioxide or the ammonium and, thus, exhibited an enhanced selectivity with respect to the detection of ammonia. The amount of ammonia detected by the ammonia sensor of the present invention demonstrated an essentially linear correlation with respect to known amounts of ammonia as measured by Cobas. Further, the tests results showed that the color changing reaction in the presence of ammonia is fully reversible.

Detection Accuracy

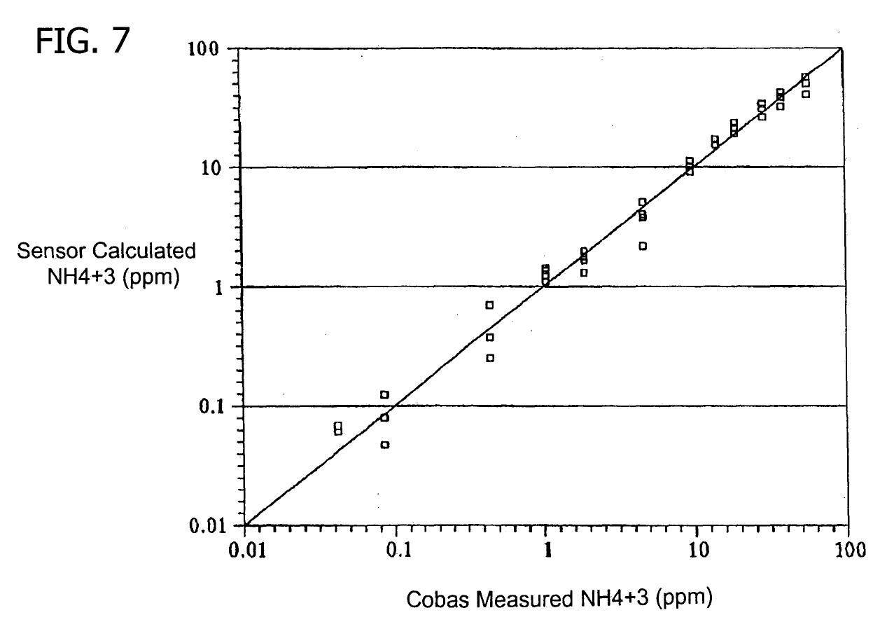

The sensors of the present invention were tested to demonstrate the accuracy and sensitivity of the ammonia detection capabilities of the present invention. The test results showed that the ammonia sensor of the present invention accurately detected amounts of ammonia in a test solution ranging from about 0.01 ppm to about 800 ppm. The determination was based on a correlation (R2=0.8635) of the amount of ammonia detected by the sensors of the present invention versus the known amounts of ammonia in the test solutions ranging from about 0.1 ppm to about 100 ppm as measured by a chemical analyzer (e.g., Cobas Mira) as shown in FIG. 7.

What is claimed is:

1. A method for detecting in the gastrointestinal tract of a subject the presence of a bacteria which when present in the gastrointestinal tract of the subject is associated with the catalytic degradation of urea to ammonia and carbon dioxide, the method comprising:
(a) delivering a source of urea to the gastrointestinal tract of the subject,
(b) obtaining a fluid sample from the subject after the delivery of the urea source,
(c) combining the fluid sample with an aqueous solution,
(d) contacting the aqueous solution with a sensor after the aqueous solution has been combined with the fluid sample, the sensor comprising a porous hydrophobic polymer having a dye embedded within the pores but not on the exposed surface thereof, the dye being capable of being deprotonated and undergoing a color change in the presence of ammonia, the pores being permeable to ammonia gas derived from the aqueous solution but impermeable to the aqueous solution whereby ammonia gas derived from the aqueous solution can permeate the pores and deprotonate the dye to effect a color change, and
(e) detecting a color change in the sensor after the sensor is contacted with the fluid sample.

2. The method of claim 1 wherein the bacteria causes a gastrointestinal associated disorder in the subject, the gastrointestinal associated disorder being selected from the group consisting of gastritis, peptic ulceration, gastric cancer, non-ulcer dyspesia, duodenal ulcers, gastric ulcers, duodenitis, gastric non-Hodgkin's lumphomas, intestinal metaplasia, adenocarcinoma, and esophagitis.

3. The method of claim 1 wherein the bacteria is a Helicobacter.

4. The method of claim 1 wherein the bacteria is Helicobacter pylori.

5. The method of claim 1 wherein the fluid sample is selected from the group consisting of a breath sample, a saliva sample, a perspiration vapor sample and a gastric reflux sample.

6. The method of claim 1 wherein the fluid sample is a breath sample.

7. The method of claim 6 wherein the breath sample is an exhaled sample or multiple breath samples.

8. The method of claim 1 wherein the aqueous solution further comprises sodium hydroxide.

9. The method of claim 1 wherein a droplet of water placed on the surface of the hydrophobic polymer has a contact angle of not less than about 100 degrees.

10. The method of claim 1 wherein the polymer has an average pore size of less than about 9 microns.

11. The method of claim 10 wherein the polymer has an average pore size of about 1.0 microns to about 2.5 microns.

12. The method of claim 1 wherein the polymer is selected from the group consisting of polytetrafluoroethylene, polyvinylidene difluoride, acrylic-based polymers, ethylene propylene polymers, polycarbonate, polyproylene, polyvanilidine chloride, dimethyl polysiloxane.

13. The method of claim 1 wherein the polymer comprises polyvinylidene difluoride.

14. The method of claim 1 wherein the dye is selected from the group consisting of bromophenol blue, bromothymol, phenol red, methyl orange, methyl yellow, 2,4-dinitrophenol, 2,6-dinitrophenol, and cresol red.

15. The method of claim 1 wherein the dye is bromophenol blue.

16. The method of claim 1 wherein the sensor changes color within about 5 to about 60 seconds after the porous polymer is permeated with the ammonia gas.

17. The method of claim 1 wherein the subject is administered a mouth wash comprising an antibacterial agent prior to collection of the breath sample to reduce the number of bacteria present in the subject's mouth.

18. The method of claim 1 wherein the concentration of ammonia gas is determined by measuring the color change of the dye with an optical reader.

19. The method of claim 1 wherein the sensor is in the form of a membrane.

20. The method of claim 1 wherein the sensor is in the form of particulate matter.

21. A method to detect the presence of a bacteria in the gastrointestinal tract of a subject, which when present in the gastrointestinal tract of the subject is associated with the catalytic degradation of urea to ammonia and carbon dioxide, the method comprising:
(a) delivering a source of urea to the gastrointestinal tract of the subject,
(b) obtaining a liquid sample from the subject after the delivery of the urea source,
(c) contacting the liquid sample or a derivative thereof with a sensor, the sensor comprising a hydrophobic polymeric material and a dye associated with the polymeric material, the dye having the capacity to become deprotonated and undergo a color change in the presence of ammonia,
(d) controlling the contact conditions such that the sensor responds to the presence of ammonia in the fluid sample but not to the pH of the fluid sample by undergoing an optically discernible color change, and
(e) optically detecting a color change in the sensor after the sensor is contacted with the fluid sample.

22. The method of claim 21 wherein the liquid sample is selected from the group consisting of a saliva sample and a tear sample.

23. A method to detect the presence of a bacteria in the gastrointestinal tract of a subject, which when present in the gastrointestinal tract of the subject is associated with the catalytic degradation of urea to ammonia and carbon dioxide, the method comprising:
(a) delivering a source of urea to the gastrointestinal tract of the subject,
(b) inducing the subject to exhale a breath sample into a container, the container comprising an inlet, a vent, and a sensor, and being adapted to restrict the flow of gas out of the inlet, the sensor comprising a hydrophobic polymeric material and a dye associated with the polymeric material, the dye having the capacity to become deprotonated and undergo a color change in the presence of ammonia,
(d) controlling the contact conditions such that the sensor responds to the presence of ammonia in the breath sample but not to the pH of the breath or any liquid in the container by undergoing an optically discernible color change, and
(e) optically detecting a color change in the sensor after the subject exhales into the container.

24. The method of claim 23 wherein a droplet of water placed on the surface of the hydrophobic polymer has a contact angle of not less than about 100 degrees.

25. The method of claim 23 wherein the polymer has an average pore size of less than about 9 microns.

26. The method of claim 23 wherein the polymer has an average pore size of about 1.0 microns to about 2.5 microns.

27. The method of claim 23 wherein the polymer is selected from the group consisting of polytetrafluoroethylene, polyvinylidene difluoride, acrylic-based polymers, ethylene propylene polymers, polycarbonate, polyproylene, polyvanilidine chloride, dimethyl polysiloxane.

28. The method of claim 23 wherein the polymer comprises polyvinylidene difluoride.

29. The method of claim 23 wherein the dye is selected from the group consisting of bromophenol blue, bromothymol, phenol red, methyl orange, methyl yellow, 2,4-dinitrophenol, 2,6-dinitrophenol, and cresol red.

30. The method of claim 23 wherein the dye is bromophenol blue.

31. A breath sampler for use in detecting the presence of H. pylori in the gastrointestinal tract, the breath sampler comprising a breath handler and a detection unit including a sensor capable of reacting to the presence of an indicator in the breath sample of H. pylori in the gastrointestinal tract, the breath handler being adapted to route air through the breath sampler upon inhalation by the subject to the subject while substantially blocking flow of air through the detection unit to the subject, and being adapted upon exhalation of the subject to route exhaled air through the breath handler to the detection unit for detection of H. pylori in the gastrointestinal tract.

32. A breath sampler as set forth in claim 31 wherein air flow through the detection unit is substantially blocked except upon exhalation by the subject through the breath sampler.

33. A breath sampler as set forth in claim 32 wherein the detection unit is substantially isolated from ambient except upon exhalation by the subject through the breath sampler.

34. A breath sampler as set forth in claim 32 wherein the breath handler includes an air intake, a breath sample opening and a breath sample outlet, the detection unit being arranged to receive air from the breath handler by way of the breath sample outlet, the breath handler receiving ambient air through the air intake upon inhalation by a subject and directing air to the breath sample opening for inhalation by the subject, and blocking a path from the breath sample outlet to the breath sample opening, the breath handler directing exhaled air from the subject to the breath sample outlet and thence to the detection unit, and blocking a path from the breath sample opening to the air intake.

35. A breath sampler as set forth in claim 34 wherein the breath handler comprises a tube having the air intake at one end thereof and the breath sample opening at an opposite end thereof, and a collection branch in fluid communication with the tube and the detection unit and including the breath sample outlet.

36. A breath sampler as set forth in claim 35 wherein the breath handler further comprises a first valve located generally at the air intake end of the tube for permitting inhaled air to flow into the tube but blocking the breath sample from flowing out of the tube through the air intake, and a second valve located in the collection branch for permitting the breath sample to flow from the tube through the collection branch and into the detection unit and blocking flow of air from the detection unit through the collection branch into the tube.

37. A breath sampler as set forth in claim 36 wherein the detection unit includes a vent for exhausting excess exhaled air from the detection unit, and wherein the detection unit further comprises a third valve disposed for permitting exhaust of exhaled air under pressure through the vent from the detection unit and inhibiting the intake of ambient air through the vent from outside the detection unit.

38. A breath sampler as set forth in claim 37 wherein the first, second and third valves are one way valves biased to a closed position in the absence of an air pressure differential across the valves.

39. A breath sampler as set forth in claim 31 further comprising liquid in the detection unit, the breath handler being adapted to discharge the breath sample into the liquid.

40. A breath sampler as set forth in claim 31 wherein the detection unit includes a vent for exhausting excess gas from the detection unit, and the breath handler comprises a tube having an intake end including an air intake and a mouthpiece portion including a breath sample opening, the intake end being positioned to locate the air intake out of registration with the vent to inhibit inhalation of exhausted air from the detection unit.

41. A breath sampler as set forth in claim 40 wherein the breath handler is pivotally mounted in the detection unit.

42. A breath sampler as set forth in claim 31 wherein the breath handler comprises a tube having an intake end including an air intake and a mouthpiece portion including a breath sample opening, the mouthpiece portion being tapered toward the breath sample opening to facilitate reception into the back of the mouth near the throat.

43. A breath sampler as set forth in claim 31 wherein the detection unit comprises a collection receptacle and the sensor is mounted in the collection receptacle, the sensor being adapted to change color upon the detection of said indicator in the breath sample, and the collection receptacle being at least partially transparent to permit a line of sight to the sensor.

44. A breath sampler as set forth in claim 31 wherein the breath sample outlet is arranged relative to the detection unit for directing the breath sample against the sensor.

45. A breath sampler as set forth in claim 31 in combination with a reader arranged for reading the sensor of the detection unit and displaying a readout corresponding to the sensor indication, the reader having a self contained power source and being adapted for connection together with the breath handler and detection unit when used by the subject to obtain a breath sample.

46. The combination as set forth in claim 45 wherein the reader is formed with a handle for holding the reader, detection unit and breath handler as the breath sample is being obtained.

47. The combination as set forth in claim 46 further in combination with a supply of urea in the form of at least one of: a bottle of urea and at least one tablet of urea.

48. A breath sampler for use in detecting the presence of H. pylori in the gastrointestinal tract, the breath sampler comprising a breath handler for receiving a breath sample from a subject, and a detection unit operatively connected to the breath handler for receiving the breath sample from the breath handler, the detection unit comprising a collection receptacle, a sensor membrane disposed in the receptacle capable of reacting to the presence of ammonia in the breath sample indicating the presence of H. pylori in the gastrointestinal tract, and a liquid in the collection receptacle generally covering the membrane, the liquid being capable of capturing ammonia from the breath sample to retain it in proximity to the sensor membrane.

49. A breath sampler as set forth in claim 48 wherein the liquid is water.

50. A breath sampler as set forth in claim 48 wherein the collection receptacle includes a bottom wall, the sensor membrane being disposed on the bottom wall and covered by the liquid.

51. A breath sampler for use in detecting the presence of H. pylori in the gastrointestinal tract, the breath sampler comprising a breath handler for receiving a breath sample from a subject, the breath handler including a mouthpiece portion having a breath sample opening in a distal end thereof, the mouthpiece portion being shaped for reception in a subject's mouth to locate the breath sample opening proximate the throat, and a detection unit operatively connected to the breath handler for receiving the breath sample from the breath handler, the detection unit including a sensor for detecting an indicator present in the breath sample of H. pylori in the gastrointestinal tract.

52. A breath sampler as set forth in claim 51 wherein the mouthpiece portion tapers in at least one dimension toward the breath sample opening.

53. A breath sampler as set forth in claim 52 wherein the mouthpiece portion tapers in cross sectional width and height.

54. A breath sampler as set forth in claim 53 wherein the breath handler comprises a tube including the mouthpiece portion and breath sample opening, and a collection branch extending from the tube to the detection unit, the mouthpiece portion extending from the collection branch to the breath sample opening and having a length selected to extend to the back of the subject's mouth.
